(19) United States
(12) Patent Application Publication
(10) Pub. No.: US 2007/0160547 A1
(43) Pub. Date: Jul. 12, 2007

(54) Method of applying a composition

(76) Inventors: Janet Duffy, Wappingers Falls, NY (US); John Nicholson, Ramsey, NJ (US); Roy U. Rojas Wahl, Teaneck, NJ (US)

Correspondence Address: DILWORTH & BARRESE, LLP, 333 EARLE OVINGTON BLVD., SUITE 702, UNIONDALE, NY, 11553, US

(21) Appl. No.: 11/329,626

(22) Filed: Jan. 11, 2006

(51) Int. Cl. A61K 8/89; A61K 8/49; A61K 8/00; A61K 31/74; A61K 8/18

(52) U.S. Cl. 424/59; 424/70.12; 424/78.08

(57) ABSTRACT

There is provided herein, in one specific embodiment, a method of applying a trisiloxane-containing composition to the surface of a substrate comprising: applying to a surface of a substrate a composition comprising a trisiloxane of the general formula (1):
MDM (1) wherein
M=R^{A}R^{B}R^{C}SiO_{1/2},
D=R^{D}R^{F}SiO_{1/2}
   in which R^{D} and R^{F} each independently is a monovalent, divalent or trivalent hydrocarbon radical possessing from 2 to 8 carbon atoms or a monovalent, divalent or trivalent hydrocarbon radical possessing from one to sixty carbon atoms other than from 2 to 8 carbon atoms, provided that at least one of R^{D} and R^{F} is a monovalent, divalent or trivalent hydrocarbon radical possessing from 2 to 8 carbon atoms, and R^{A}, R^{B}, R^{C} each independently is a monovalent, divalent or trivalent hydrocarbon radical possessing from one to sixty carbon atoms other than from 2 to 8 carbon atoms.

### BACKGROUND OF THE INVENTION

(1) Field of the Invention

The invention relates to a method of applying a composition.

(2) Description of Related Art

Many industries and markets thrive on being able to deliver multiple performance products based on mixtures of several components, with each component having a performance and compatibility profile, which can lend that component to properties that are important to or desirable in certain final product formulations. In methods of applying compositions some desirable properties can be derived from the use of cyclic siloxanes in the formulation. Although cyclic siloxanes can provide some of these desired properties, they are low viscosity, highly flowable liquids. Thus, they are not easily held in a formulation, preferring rather to separate and flow out of a given container or flow uncontrollably across the skin and/or other substrate when used in a specific application.

Cyclic siloxanes have limitations in terms of their performance and compatibility profile. A desired performance and/or compatibility profile possessing the various desirable properties is not always achievable by using a cyclic siloxane. Certain applications can require different properties that cannot be met by the use of cyclic siloxanes.

For example, one of the disadvantages of the current state of the art is that many sprayable formulations suffer from clogging of the spraying system, poor or coarse droplet formation and uneven or poor coverage with too much run-down on a non-horizontal surface. Run-down can, for example, lead to a disadvantageous distribution of benefit ingredients delivered onto any substrate, transporting those ingredients to areas where they might be undesired and/or leading to an uneven distribution where they are desired. Also, clogging of spraying nozzles can impose too much mechanical stress and lead to early wear down of spraying systems. Coarse droplets will lead to uneven actives distribution. The resulting poor coverage will in turn lead to lower performing attributes. Often ethanol is used as a dispersant, however, in certain applications such as personal care it suffers from a concomitant drying effect that can cause skin flakiness and irritation. Therefore there exists a need for a sprayable formulation without the disadvantages mentioned above.

### BRIEF DESCRIPTION OF THE INVENTION

In this brief description it is noted that the present inventors have unexpectedly discovered, in one specific embodiment, a method of applying a trisiloxane-containing composition, said trisiloxane-containing composition comprising specific trisiloxane(s) that can provide greatly improved physical properties to trisiloxane-containing composition(s).

In one embodiment herein there is provided a method of applying a trisiloxane-containing composition to the surface of a substrate comprising:
applying to a surface of a substrate a composition comprising a trisiloxane of the general formula (1):
   MDM (1)
   wherein
M=R^{A}R^{B}R^{C}SiO_{1/2},
D=R^{D}R^{F}SiO_{1/2}
   in which R^{D} and R^{F} each independently is a monovalent, divalent or trivalent hydrocarbon radical possessing from 2 to 8 carbon atoms or a monovalent, divalent or trivalent hydrocarbon radical possessing from one to sixty carbon atoms other than from 2 to 8 carbon atoms, provided that at least one of R^{D} and R^{F} is a monovalent, divalent or trivalent hydrocarbon radical possessing from 2 to 8 carbon atoms, and R^{A}, R^{B}, R^{C} each independently is a monovalent, divalent or trivalent hydrocarbon radical possessing from one to sixty carbon atoms other than from 2 to 8 carbon atoms.

### BRIEF DESCRIPTION OF THE DRAWINGS

FIGS. 1-4 are xerographic reproductions of photographs of patterns resulting from the application of compositions within (Examples I and II) and other compositions outside (Comparative Examples I and II) the scope of the disclosure herein, said photographs being taken shortly after (1-6 seconds) application of said compositions.

### DETAILED DESCRIPTION OF THE INVENTION

Surprisingly, applicants have discovered, in one specific embodiment a method of applying a trisiloxane-containing composition, wherein said trisiloxane-containing composition comprises certain trisiloxanes, which provide superior physical properties to those trisiloxane-containing compositions, leading to the herein noted advantageous properties such as reduced run-down, to applications and/or formulations containing the trisiloxane-containing composition when sprayed onto a surface of a substrate.

As used herein the terms polyorganosiloxane and organopolysiloxane are interchangeable one with the other.

As used herein the terms formulation and application are interchangeable with one another.

It will be understood herein that all uses of the term centistokes were measured at 25 degrees celsius.

It will be understood herein that all specific, more specific and most specific ranges recited herein comprise all sub-ranges therebetween.

It will be understood herein that all percent ranges are weight percent based upon total weight of a formulation comprising trisiloxane-containing composition unless stated otherwise.

The expression “run-down” shall be understood herein to mean the extent of visually observed downward flow of trisiloxane-containing composition applied to cellulose paper, run-down being measured on a scale of 1 to 3 as follows:
0=no downward extension of the incident spray
1=little downward extension of the incident spray;
2=readily discernible, but moderate, downward extension of the incident spray; and
3=clearly evident, and extensive downward extension of the incident spray.

In one specific embodiment herein trisiloxane-containing composition, can contain specifically less than about 100 weight percent, more specifically less than about 10 weight percent, and most specifically less than about 1 weight percent water based on the total weight of trisiloxane-containing composition. In one more specific embodiment trisiloxane-containing composition can contain no water, so that trisiloxane-containing composition contains no atmospheric water and/or added water.

In another specific embodiment herein substrate can comprise skin, hair, nail(s), eyelashe(s), eyebrow(s), lip(s), leave(s) of plant(s), paper, leather, metal, plastic, fiber(s), glass, fabric(s), wood, woven(s) and non-woven(s) and combinations thereof.

In yet another more specific embodiment, the hydrocarbon radical(s) of trisiloxane of the general formula (1) can be any hydrocarbon radical commonly associated with such polymers and can generally be selected from the non-limiting examples of alkyl radicals of 1 to about 60 carbon atoms, such as methyl, ethyl, propyl; cycloalkyl radicals such as cyclohexyl, cycloheptyl, cyclooctyl; mononuclear aryl radicals such as phenyl, methylphenyl, ethylphenyl; alkenyl radicals such as vinyl and allyl; alkylene oxide radicals, such as ethylene oxide, propylene oxide and mixtures thereof, and haloalkylradicals such as 3,3,3, trifluoropropyl. In a more specific embodiment, the hydrocarbon radicals of trisiloxane of general formula (1) are alkyl radicals of 1 to 8 carbon atoms, and are most specifically methyl or ethyl. In one embodiment, trisiloxane of general formula (1) has some hydroxyl groups in the polymer. In one other specific embodiment the hydrocarbon radical(s) of trisiloxane of general formula (1) can be any of the organic functional groups described in U.S. Pat. No. 6,531,540 the contents of which are incorporated herein by reference in its entirety. It will be understood that the nomenclature used in U.S. Pat. No. 6,531,540 regarding hydrocarbon radicals will deemed to be equivalent to hydrocarbon radical(s) as they are described herein, in that any hydrocarbon radical described in U.S. Pat. No. 6,531,540 can be used as hydrocarbon radical as is described herein so that the terms hydrocarbon radical as it is described in U.S. Pat. No. 6,531,540 and hydrocarbon radicals as used herein is interchangeable. In one embodiment herein, it will be understood that any hydrocarbon radical described herein can be present on trisiloxane of general formula (1) or can be bonded to said trisiloxane through a reaction with an organic compound, such as the non-limiting example of an olefin, specifically an alpha-olefin.

In one specific embodiment herein trisiloxane of the general formula (1) can be made by any known process. In one more specific embodiment, trisiloxane-containing composition is an alkyl-substituted trisiloxane as described by general formula (1) above. In one embodiment, the term “alkyl” means a saturated straight or branched monovalent hydrocarbon radical, such as the hydrocarbon radicals described above. In a specific embodiment, monovalent alkyl groups are selected from linear or branched alkyl groups containing from 1 to 60 carbon atoms per group, such as the non-limiting examples of, methyl, ethyl, propyl, iso-propyl, n-butyl, iso-butyl, sec-butyl, tert-butyl, pentyl, hexyl, heptyl, decyl, and dodecyl. In another specific embodiment trisiloxane of general formula (1) is linear alkyltrisiloxane which is high purity material which results from the alkylation of a polyorganosiloxane having the formula (A):
M¹D²M¹ (A)
where
M¹=R^{G}R^{J}R^{K}SiO_{1/2}; and
D²=R^{L}R^{M}SiO_{2/2};
   where R^{G}, R^{J}, R^{K}, R^{L} and R^{M} are independently monovalent hydrocarbon radicals having from one to sixty carbon atoms or hydrogen, provided that at least one of R^{G}, R^{J}, R^{K}, R^{L} and R^{M} are hydrogen. In a more specific embodiment R^{L} and/or R^{M} is hydrogen. In one embodiment, a high purity material as described herein is defined as substantially comprising the alkylated polyorganosiloxane of formula (A). In a specific embodiment herein, alkylation of a polyorganosiloxane having the formula (A) can be accomplished by reacting a polyorganosiloxane having the formula (A) with alkene compound such as the non-limiting example of ethylene. In a more specific embodiment a high purity material comprises greater than about 90 weight percent of the alkylated polyorganosiloxane of formula (A) based on the total weight of high purity material. In an even more specific embodiment a high purity material comprises greater than about 95 weight percent of the alkylated polyorganosiloxane of formula (A) based on the total weight of the high purity material. In a most specific embodiment a high purity material comprises greater than about 97 weight percent of the alkylated polyorganosiloxane of formula (A) based on the total weight of the high purity material. In another specific embodiment herein, trisiloxane of general formula (1) can be made as a result of an alkylation of a polyorganosiloxane as described in U.S. patent application Ser. No. 11/274,450 filed on Nov. 15, 2005, the contents of which are incorporated by reference herein in their entirety. In one other embodiment herein trisiloxane of general formula (1) can be formed by any known or conventionally used method known to those skilled in the art.

In a more specific embodiment herein trisiloxane of the general formula (1) can more specifically comprise where R^{D} and/or R^{F} is a monovalent, divalent or trivalent hydrocarbon radical possessing from 2 to 6 carbon atoms. In an even more specific embodiment herein trisiloxane of the general formula (1) can comprise where R^{D} and/or R^{F} is a monovalent, divalent or trivalent hydrocarbon radical possessing from 2 to 4 carbon atoms. In yet an even more specific embodiment trisiloxane of the general formula (1) can even more specifically comprise where R^{D} and/or R^{F} is ethyl. In a most specific embodiment trisiloxane of the general formula (1) can comprise where R^{D} is ethyl and R^{F} is methyl.

In one specific embodiment herein some non-limiting examples of trisiloxane of general formula (1) are linear alkyltrisiloxanes selected from the group consisting of ethyltrisiloxane, such as the non-limiting examples of 1,1,1,3,5,5,5-heptamethyl-3-ethyltrisiloxane; octyltrisiloxane, such as the non-limiting example of 1,1,1,3,5,5,5-heptamethyl-3-octyltrisiloxane; hexyltrisiloxane such as the non-limiting example of 1,1,1,3,5,5,5-heptamethyl-3-hexyltrisiloxane, and combinations thereof. In one specific embodiment herein trisiloxane of general formula (1) can be at least one trisiloxane such as those described in U.S. Patent Application Publication No. 2004/0197284A1 which is incorporated by reference herein in its entirety. In another specific embodiment herein trisiloxane of the general formula (1) can be at least one trisiloxane such as those described in U.S. Patent Application Publication No. 2005/0069564A1 the contents of which are incorporated by reference herein in its entirety. In yet one even more specific embodiment, trisiloxane of the general formula (1) can be selected from the group consisting of 3-pentyl-1,1,1,3,5,5,5-heptamethyltrisiloxane; 1,1,1,3,5,5,5-heptamethyl-3-octyltrisiloxane, sold, for example, under the name “Silsoft 034” by the company General Electric; 1,1,1,3,5,5,5-heptamethyl-3-hexyltrisiloxane; 3-(1-ethylbutyl)-1,1,1,3,5,5,5-heptamethyltrisiloxane; 1,1,1,3,5,5,5-heptamethyl-3-(1-methylpentyl)trisiloxane; 1,1,1,3,5,5,5-heptamethyl-3-(1-methylpropyl)trisiloxane; 3-(1,1-dimethylethyl)-1,1,1,3,5,5,5-heptamethyltrisiloxane; 1,1,1,5,5,5-hexamethyl-3,3-bis(1-methylethyl)trisiloxane; 3-(3,3-dimethylbutyl)-1,1,1,3,5,5,5-heptamethyltrisiloxane; 1,1,1,3,5,5,5-heptamethyl-3-(3-methylbutyl)trisiloxane; 1,1,1,3,5,5,5-heptamethyl-3-(3-methylpentyl)trisiloxane; 1,1,1,3,5,5,5-heptamethyl-3-(2-methylpropyl)trisiloxane; 1,1,1,3,5,5,5-heptamethyl-3-propyltrisiloxane; 3-isohexyl-1,1,1,3,5,5,5-heptamethyltrisiloxane; 3-tert-pentyl-1,1,1,3,5,5,5-heptamethyltrisiloxane; 3-neo-pentyl-1,1,1,3,5,5,5-heptamethyltrisiloxane; 1,1,1,5,5,5-hexamethyl-3,3-dipropyltrisiloxane; 3,3-diethyl-1,1,1,5,5,5-hexamethyltrisiloxane; 3,3-dibutyl-1,1,1,5,5,5-hexamethyltrisiloxane; 3-ethyl-1,1,1,3,5,5,5-heptamethyltrisiloxane; 3-heptyl-1,1,1,3,5,5,5-heptamethyltrisiloxane; 1,1,1,5,5,5-hexamethyl-3,3-diethyltrisiloxane; 1,1,1,3,5,5,5-heptamethyl-3-butyltrisiloxane and combinations thereof.

In one specific embodiment trisiloxane of the general formula (1) can comprise two or more trisiloxanes of the general formula (1) as described herein.

The amount of trisiloxane of the general formula (1) as described above used in trisiloxane-containing composition will vary depending on the intended use of the trisiloxane-containing composition, the other components, the desired result, and the like considerations, and can be readily determined by one of ordinary skill in the industry specific arts. In another specific embodiment the trisiloxane- of the general formula (1) is present in the trisiloxane-containing composition in an amount of specifically of from about 0.001 to about 99 weight percent based on the total weight of the trisiloxane-containing composition, more specifically of from about 0.01 to about 85 weight percent based on the total weight of the trisiloxane-containing composition, even more specifically of from about 0.1 to about 50 weight percent based on the total weight of the trisiloxane-containing composition and most specifically of from about 0.2 to about 20 weight percent based on the total weight of the trisiloxane-containing composition. In yet an even further specific embodiment the trisiloxane of the general formula (1) is present in the trisiloxane-containing composition in an amount of specifically of from about 10 to about 2 weight percent based on the total weight of the trisiloxane-containing composition.

Specific formulations comprising trisiloxane-containing composition described herein will vary depending on industry and will be determined by a person skilled in the art of the industry. In yet another specific embodiment herein trisiloxane-containing composition further comprises formulation component in an amount of specifically of from about 99 to about 0.001 weight percent based on the total weight of the trisiloxane-containing composition, more specifically of from about 85 to about 0.01 weight percent based on the total weight of the trisiloxane-containing composition, even more specifically of from about 50 to about 0.1 weight percent based on the total weight of the trisiloxane-containing composition and most specifically of from about 20 to about 2 weight percent based on the total weight of the trisiloxane-containing composition. In yet an even further specific embodiment trisiloxane-containing composition further comprises formulation component in an amount of specifically of from about 10 to about 2 weight percent based on the total weight of the trisiloxane-containing composition.

In one embodiment formulation component can comprise conventional and known formulation components as they are known in the various industries by those skilled in the art. In yet even a further specific embodiment herein formulation component is selected from the group consisting of propellant, surfactant, emulsifier, solvent, emollient, moisturizer, humectant, pigment, colorant, fragrance, biocide, preservative, antioxidant, anti-microbial agent, anti-fungal agent, insecticide, paint, antiperspirant agent, aerosol-driver, exfoliants, alcohol, hormone, enzyme, medicinal compound, vitamin, alpha-hydroxy acid, beta-hydroxy acid and/or other hydroxy acid, retinol and its derivatives, niacinamide, skin lightening agent, salt, electrolyte, polyol, absorbing agent for ultraviolet radiation, botanical extract such as the non-limiting examples of herbal extracts and tea tree oils, organic oil, wax, film former, thickening agent, particulate filler, clay, plasticizer, occlusive, sensory enhancer, ester, resin and film formers, spherical or other optically active particle including the non-limiting examples of silicone spheres, and combinations thereof.

In another specific embodiment there are provided combinations of the foregoing trisiloxane of the general formula (1) with other types of silicones selected from the group consisting of silicone oil, silicone gum, silicone solid, silicone emulsion, silicone resin, silicone copolyol, alkyl silicones, silicone waxes, silicone amines, silicone ammonium salts, quaternary silicone amines, spherical silicones, silicone gels, silicone crosspolymer networks, polyether silicones, and combinations thereof. The relative amounts of each of the silicones are adjusted so as to optimize the industry specific characteristics of the trisiloxane-containing composition, for example reduced run-down in, specifically for, but not limited to, sprayable personal care compositions. In a further specific embodiment herein, trisiloxane-containing composition can further comprise silicone fluid other than said trisiloxane of the general formula (1). In a more specific embodiment, said silicone fluid other than trisiloxane of general formula (1) can be cyclic siloxane and/or linear silicone fluid such as, decamethylcyclopentasiloxane fluid and/or high molecular weight polyorganosiloxane fluid, respectively. In a most specific embodiment herein said high molecular weight polyorganosiloxane fluid has a greater viscosity relative to trisiloxane of the general formula (1) and can be selected from the group consisting of organo polydimethylsiloxanes having a viscosity of specifically from about 1 to about 400, more specifically of from about 3 to about 350, and most specifically from about 5 to about 300 centistokes, wherein the organo groups are any of the above described hydrocarbon radicals. In one specific embodiment organo polydimethylsiloxane can have a viscosity of from about 1 to about 1000 centistokes, more specifically 2 to about 500 centistokes and most specifically 5 to about 10 centistokes. In one very specific embodiment, polydimethylsiloxane can have a viscosity of 5 centistokes. In one very specific embodiment, polydimethylsiloxane can have a viscosity of 50 centistokes.

In one embodiment herein, trisiloxane-containing composition as described herein, can be utilized as prepared or as the silicone component in emulsions, such as are described in U.S. Pat. Nos. 6,531,540; 6,060,546 and 6,271,295, the contents for all of these patents are incorporated by reference herein in their entirety.

In one further embodiment herein, precious metal catalysts suitable for making trisiloxane of general formula (1) and silicone fluid other than trisiloxane of general formula (1) described herein are well known in the art and include complexes of rhodium, ruthenium, palladium, osmium, iridium and/or platinum. For example, many types of platinum catalysts for this SiH olefin addition reaction are known, including Karstedt's catalyst, and such platinum catalysts may be used to generate the compositions of the present invention. Additional background concerning the art may be found in J. L. Spier, “Homogeneous Catalysis of Hydrosilation by Transition Metals”, in Advances in Organometallic Chemistry, volume 17, pages 407 through 447, F. G. A. Stone and R. West editors, published by Academic Press (New York, 1979). Those skilled in the art can easily determine an effective amount of platinum catalyst. Generally an effective amount ranges from about 0.1 to about 50 parts per million of the total trisiloxane-containing composition. In a more specific embodiment an effective amount more specifically ranges from about 1 to about 20 parts per million of the total trisiloxane-containing composition and most specifically from about 4 to about 15 parts per million of the total trisiloxane-containing composition.

In yet an even further specific embodiment herein, applying trisiloxane-containing composition comprises using a spray that employs at least one spraying, dispensing, diffusing or mistyfying process. In an even further specific embodiment said spraying, dispensing, diffusing or mistyfying processes can be any such conventionally known processes including but not limited by industry typical flow rates, droplet sizes and pressure build-ups.

In yet still an even further specific embodiment applying the trisiloxane-containing composition comprises using a spray selected from the group consisting of a fragrance and lotion pump, fine mist sprayer, a specialty pump, an anti-clog pump, a spray-through cap, a nozzle, an airless pump, an airless dispenser, a dual dispensing pump, a fine mist pump, a trigger pump, a foam pump, a trigger sprayer, an aerosol sprayer, a squeeze roamer and combinations thereof to provide an application of said spray.

In one specific embodiment herein the process of spraying, dispensing, diffusing or mystifying can comprise a swift sudden stream of ejected liquid or fluid, which includes the non-limiting examples of: a) the process of atomization, i.e. the reduction of infinitesimal particles, such as of a fluid or emulsion, to a fine spray or mist, often by passing the particles through a nozzle; b) the process of sprinkling including irrigation and fire sprinkling, i.e. the distribution of water by spraying it into the air; c) the process of jet streaming, i.e. a stream of fluid produced by discharge through an orifice or small opening and/or nozzle, where nozzle is a mechanical device to control the characteristics of a fluid flow as it exits from an enclosed chamber into some medium. Often here, the kinetic energy of the flowing medium is increased at the expense of a certain pressure energy of the fluid before passing through the nozzle.

In one further specific embodiment applying trisiloxane-containing composition further comprises spraying specifically from about 0.001 milliliters to about 50 milliliters per application of spray, more specifically from about 0.01 milliliters to about 5 milliliters per application of spray and most specifically from about 0.1 milliliters to about 0.5 milliliters per application of spray.

In yet a further specific embodiment applying the trisiloxane-containing composition further comprises spraying at a distance of specifically of from about 0.01 centimeters to about 10,000 centimeters from a surface of a substrate, more specifically of from about 0.1 centimeters to about 100 centimeters from a surface of a substrate and most specifically of from about 1 centimeters to about 50 centimeters from a surface of a substrate.

In one further embodiment herein method of applying trisiloxane-containing composition further comprises where said trisiloxane-containing composition comprises other than a mold release composition, a cosmetic spray, a gel or an emulsion.

In one specific embodiment herein the method of applying trisiloxane-containing composition can comprise where the trisiloxane-containing composition further comprises the silicone gels described in U.S. patent application Ser. No. 11/274,450 filed on Nov. 15, 2005, the contents of which are incorporated herein in their entirety.

In another specific embodiment herein, the method of applying trisiloxane-containing composition can further comprise where said trisiloxane-containing composition exhibits properties that are improved over a sprayable composition containing other than the composition of general formula (1). In a more specific embodiment method of applying trisiloxane-containing composition where trisiloxane-containing composition exhibits properties that are improved over a sprayable composition containing other than the composition of general formula (1) can comprise where other than the composition of general formula (1) can comprise volatile siloxane, such as the non-limiting example of cyclic siloxane. Cyclic siloxanes that are used as volatile siloxanes are well known in the prior art and will not be described in detail herein. In an even more specific embodiment herein, said properties that are improved over a sprayable composition containing other than the composition of general formula (1) are at least one selected from the group consisting of reduced run-down, better coverage, improved actives distribution and/or performance, rapid drying, less over-wetting, easy formulation, non-tackiness, improved spreadability, improved homogeneity, improved organic compatibility, reduced skin irritation, improved active delivery, improved skin feel, enhanced SPF, less or more shine as desired, smaller droplet size, and less mechanical stress on the spraying system employed. The improvements of any one or more of the foregoing properties may be synergistic. For example, fast drying can lead to less over-wetting, which, in some instances, can lead to improved mechanical stability of the substrate, for example, but not limited to, the paper industry, where a wet paper has less mechanical strength than a dry paper. In another advantageous feature, the method of applying trisiloxane-containing composition herein can advantageously reduce the run-down of sprayable formulations applied in many different industries, for example in sprayable cosmetic or personal care formulations, by the merely adding trisiloxane-containing composition described herein to such formulations.

In one specific embodiment herein the above described formulation components do not adversely affect or substantially adversely affect the run-down reduction or any of the above-identified improved properties of the trisiloxane-containing composition. In another specific embodiment herein the above-mentioned improved properties can be the result of a synergistic effect produced by using a synergistic component selected from the group consisting of emulsifier; preservative; polymer; thixotropic and non-thixotropic film former; sunscreening agent; sunblocking agent, colorant; pigment; fragrance; active ingredient such as the non-limiting examples of vitamin, alpha-hydroxy acid, beta-hydroxy acid and/or other hydroxy acid; retinol and its derivatives; niacinamide; tocopherol and/or its derivatives, skin lightening agent; humectant; occlusive; sensory enhancer; ester; other resin and film former; spherical or other optically active particle including silicone sphere; as well as other silicone; emollient; surfactant; and combinations thereof.

In another specific embodiment the method of applying trisiloxane-containing composition herein can be used in markets and industries selected from the group consisting of aerospace; agriculture; food & beverage; tissue & non-wovens; automotive including the non-limiting examples of automotive body panels, automotive energy management, automotive headliners, automotive headrests and armrests, automotive instrument panels, automotive seating; steering wheels; tires; coatings; inks; paints; construction including the non-limiting examples of construction sealants, discontinuous panels, flexible face panels, windows and doors, pipes; consumer including the non-limiting examples of consumer appliances, footwear, furniture, bedding, carpets; electronics, including automotive electronics; semiconductors; healthcare; homecare including the non-limiting examples of hard surface cleaners, laundry and fabric care, polishes and treatments; industrial assembly; industrial cleaning; mold making; packaging and labeling; power & electric distribution; process industries including the non-limiting examples of apparel & leather, oil & gas, pulp & paper, release; transportation including the non-limiting examples of insulated tanks, shipping containers; pool & spa; motorcycles including the non-limiting example of motor scooters, personal care and combinations thereof.

In yet another specific embodiment the method of applying trisiloxane-containing composition herein can further comprise where the personal care markets and industries can comprise using the trisiloxane-containing composition in a personal care application selected from the group consisting of deodorants, antiperspirants, antiperspirant/deodorants, in the non-limiting forms of sprays, sticks and roll-on products; shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and/or lotions, cuticle softeners, sunscreen, insect repellent, anti-aging products, lipsticks, foundations, face powders, eye liners, eye shadows, blushes, makeup, mascaras, moisturizing preparations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and/or shampoos, baby conditioners, shaving preparations, cucumber slices, skin pads, make-up removers, facial cleansing products, cold creams, sunscreen products, spritzes, paste masks and/or muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and/or lotions, oil/water, water/oil, multiple and macro and micro emulsions, pickering emulsions, multiple-phase emulsions, water-resistant creams and/or lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail- and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and/or cloths, depilatory preparations, personal care lubricants, nail coloring preparations, drug delivery systems for topical application of medicinal compositions that are to be applied to the skin and combinations thereof.

In another specific embodiment herein the method of applying trisiloxane-containing composition can comprise where trisiloxane-containing composition is used in applications where it is desired to have any coating related advantageous property including but not limited to skin protection, such as the non-limiting examples of sunscreens, hair care products, cosmetics, anti-aging products, moisturizers, skin care products and combinations thereof.

In yet another specific embodiment herein the method of applying trisiloxane-containing composition can further comprise where a personal care application is a suncare formulation that can further comprise sunscreen agent selected from the group consisting of UV blocking or UV absorbing sunscreening agent, and combinations comprising at least one of the foregoing sunscreening agents. In yet a further specific embodiment sunblocking agents are generally inorganic, such as the non-limiting examples of silicone or otherwise treated titanium dioxides, titanium dioxide, silicone or otherwise treated zinc oxides, zinc oxide. In yet still a further specific embodiment absorbing sunscreening agents which are generally organic species include the non-limiting examples of UV-A absorbers, which generally absorb radiation in the 320 to 400 nm region of the ultraviolet spectrum, such as the non-limiting examples of anthranilates, benzophenones, and dibenzoyl methanes; and UV-B absorbers, which generally absorb radiation in the 280 to 320 nm region of the ultraviolet spectrum, such as the non-limiting examples of, p-aminobenzoic acid derivatives, camphor derivatives, cinnamates, and salicylates. In one specific embodiment some non-limiting examples of organic sunscreening agents include p-aminobenzoic acid, avobenzone cinoxate, dioxybenzone, homosalate, menthyl anthranilate, octocrylene, octyl methoxycinnamate, octyl salicylate, oxybenzone, padimate, phenylbenzimidazole sulfonic acids, sulisobenzone, trolamine salicylate, aminobenzoic acid, amyldimethyl p-aminobenzoic acid, diethanolamine p-methoxycinnamate, digalloyl trioleate, 2-ethylhexyl-2-cyano-3,3-diphenylacrylate, ethylhexylp-methoxycinnamate, 2-ethylhexyl salicylate, glyceryl aminobenzoate, homomenthyl salicylate, homosalate, 3-imidazol-4-ylacrylic acid and the ethyl ester thereof, methyl anthranilate, octyldimethyl PABA, 2-phenylbenzimidazole-5-sulfonic acid and salts, sulisobenzone, triethanolamine salicylate, N,N,N-trimethyl-4-(2-oxoborn-3-ylidene methyl)anillinium methyl sulfate, aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammonium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, ca-(2-oxoborn-3-ylidene)-tolyl-4-sulfonic acid and soluble salts thereof, 3-(4′-sulfo)benzyliden-bornan-2-one and soluble salts thereof, 3-(4′-methylbenzylidene)-d,1-camphor, 3-benzylidene-d,1-camphor, benzene 1,4-di(3-methylidene-10-camphosulfonic) acid and salts thereof, urocanic acid, 2,4,6-tris-(2′-ethylhexyl-1′-oxycarbonyl)-anilinol 1,3,5-triazine, 2-(p-(tert-butylamido)anilinol-4,6-bis-(p-(2′-ethylhexyl 1′-oxycarbonyl) anilinol 1,3,5-triazine, 2,4-bis{1,4-(2-ethylhexyloxy)-2-hydroxyl-phenyl}-6-(4-methoxyphenyl)-1,3,5-triazine, the polymer of N-(2 et 4)-(2-oxoborn-3-yliden)methylbenzyl acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (Drometrizole Trisiloxane), solubilized 2,2′-methylene-bis-1,6-(2H-benzotriazol-2-yl)-4-(1,1,3,3-tetramethylbutyl)phenol, 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, and combinations comprising at least one of the foregoing sunscreening agents. In one embodiment, it has been found that a combination of at least three sunscreening agents is effective. In another embodiment it has been found that a combination of four sunscreening agents is effective, especially where chemical sunscreening agents are used. In a further embodiment, a formulation which comprises a combination of one or several sunblocking agents with one or several sunscreening agents is effective.

In one specific embodiment herein when trisiloxane-containing composition is formulated in a sunscreen formulation, the sunscreen formulation can be in the form of a lamellar emulsion, a mirocoemulsion, or a nanoemulsion or a pickering emulsion. In addition, the emulsions may be a fluid simple emulsion, a fluid multiple emulsion, a rigid simple emulsion, or a rigid multiple emulsion. The simple emulsion or multiple emulsion can comprise a continuous aqueous phase containing dispersed lipid vesicles or oil droplets, or a continuous fatty phase dispersed lipid vesicles or water droplets. In one embodiment, the sunscreen application is an emulsion having a continuous aqueous phase, and is usually in form of a sprayable composition, be it single phase, bi- or multiple-phasic, an emulsion, a gel, a lotion and the like.

In another specific embodiment herein, it will be understood that reduced “run-down” includes any phenomena where the area wetted by trisiloxane-containing composition sprayed onto a surface of a substrate has a reduced or eliminated change in shape or size. This includes gravity driven processes as well as processes driven by any other physical- or mechanical force, such as, but not limited to, capillary forces.

In yet an even further specific embodiment the method of applying trisiloxane-containing composition can further comprise where personal care application can further comprise emulsifier selected from the group consisting of non-ionionic biopolymer blends such as the non-limiting examples of sucrose palmitate (and) glyceryl stearate (and) glyceryl stearate citrate (and) sucrose (and) mannan (and) xanthan gum (arlatone V-175) from Uniquema; polyethoxylated emulsifiers such as the non-limiting examples of propylene glycol isoceth-3 acetate (Hester PHA) from Bernel or Steareth-2 or Steareth-10 (Volpo S2 and Volpo S1-) from Croda, laureth-7, laureth-4 (Sepigel® 305) available from SEPPIC; modified polyacrylates such as the non-limiting examples of glycerin (and) glycerin polyacrylate (Hispagel 200) from Cognis; oils such as the non-limiting examples of vegetable and mineral oil; animal and/or synthetic waxes such as the non-limiting examples of beeswax, paraffin, rice bran wax, candelilla wax, carnauba wax and derivatives thereof; hydrocarbon gels or bentone type gels such as the non-limiting examples of Gel SS71, Gel EA2786, Quaternium-18 Bentonite, 38 CE, Gel ISD V or Gel ISD; organosilicone emulsifiers such as the non-limiting examples of cetyl dimethicone copolyol-polyglyceryl-4-isostearate-hexylaurate (ABIL® WE 09) available from Goldschmidt Chemical Corporation, behenate dimethicone, cetyl dimethicone copolyol (ABIL® EM 90), (ABIL® EM 97), laurylmethicone copolyol (5200), cyclomethicone and dimethicone copolyol (DC 5225 C and DC 3225 C) available from GE Silicones, cyclopentasiloxane and dimethicone copolyol (GE SF 1528 or GE 1540), PEG-20/PPG-23 Dimethicone (GE Silsoft 440); and combinations thereof.

In another specific embodiment herein the method of applying trisiloxane-containing composition can comprise where trisiloxane-containing composition can be used in a product form that can be anhydrous to non-anhydrous. In a more specific embodiment said product form can be a simple solid, a particle, a solution, a gel-network-stabilized phase and combinations thereof. In an even more specific embodiment product forms can be selected from the group consisting of a gel, an emulsion, a dispersion, a sludge, a powder, a bar, a spray, a spritz, a mousse, a stick, a balm, a foundation and combinations thereof. In a further specific embodiment emulsion product form can form a single-phase, a bi-phase, a tri-phase, or can be multiple-phased, lamellar phased, and can form a macro-, micro- or nanoemulsion, and can also form a water-in-oil-in-water, an oil-in-water-in-oil emulsion, a water in silicone emulsion or a silicone in oil emulsion, or can be an anionic-, cationic-, amphoteric- or neutral emulsion, or a pickering emulsion, as well as other physical forms.

In yet still another specific embodiment herein the method of applying trisiloxane-containing composition can further comprise where trisiloxane-containing composition comprises plasticizer selected from the non-limiting group consisting of lecithin, polysorbates, dimethicone copolyol, glycols, citrate esters, glycerin, dimethicone and combinations thereof.

In yet still another embodiment herein a method of manufacture of trisiloxane-containing composition through a process or a combination of processes known to a person skilled in the art, such as any technique of applying shear, including, but not limited to, hand- or mechanical stirring, high-, medium- or ambient- or low-temperature mixing of any type, homogenization or other special procedures for achieving small droplet sizes in emulsions including, but not limited to, phase-inversion-processes involving heating as well as any cold processes.

In another embodiment there is provided a method of manufacturing the foregoing personal care applications comprises combining the above-described trisiloxane-containing composition with a personal care ingredient such as is known to those skilled in the art. Some non-limiting examples of personal care ingredients are described in U.S. patent application Ser. No. 11/274,450 filed on Nov. 15, 2005 the contents of which are incorporated herein in their entirety.

In yet still even another embodiment the disclosed personal care compositions may exhibit a variety of advantages in personal care applications. Some such non-limiting advantages are less run-down, alone or in combination with any active ingredient. Other properties of each of the personal care applications will vary depending on the characteristics of the particular trisiloxane(s) chosen, for example the type(s) of hydrocarbyl radicals present, the presence and type(s) of any substitution(s) on the silicone backbone or on the side chain(s), the molecular weight of the trisiloxane(s), and like considerations, as well as the remaining components of the sprayable personal care application. The desired properties of specific personal care applications can be specifically determined by those skilled in the art. Some specific examples of properties provided by use of the trisiloxanes herein may include reduced tackiness, enhanced spreadability, fast drying, less over-wetting, improved homogeneity, enhanced organic compatibility, improved skin feel, controlled actives delivery, improved shine, and/or reduced skin irritation. Test panels can be used to objectively determine the reduced tackiness, improved homogeneity, enhanced organic compatibility and improved skin feel. The enhanced spreadability may be determined by measuring the coefficient of friction in a sample of the personal care composition. Skin irritation may be determined by measuring the onset of erythema. Sunscreens are evaluated according to their ability to slow erythema or sunburn resulting from exposure to ultraviolet light. Shine may be determined by measuring refractive index using a gloss meter.

In yet a further embodiment herein, there is provided a personal care formulation, such as those described above, comprising silicone gel produced by any process described in U.S. patent application Ser. No. 11/274,450 filed on Nov. 15, 2005 the contents of which are incorporated herein in their entirety.

The examples below are given for the purpose of illustrating the invention of the instant case. They are not being given for any purpose of setting limitations on the embodiments described herein. All weights are weight percent based on the total weight of trisiloxane-containing composition unless stated otherwise.

### EXAMPLES

Preparation of the following trisiloxane-containing compositions in Table 1 were accomplished by hand-stirring and addition of ingredients in the order listed, with continued stirring between each addition of the next ingredient.

TABLE 1

| | Com- | | Com- | |
|---|---|---|---|---|
| | parative | | parative | Example |
| Ingredients | Example I | Example I | Example II | II |
| Diisostearoyl | 8.00 | 8.00 | 0 | 0 |
| Trimethyl- | | | | |
| olpropane | | | | |
| Siloxy Silicate | | | | |
| C12-C15 | 10.00 | 10.00 | 0 | 0 |
| Alkylbenzoate | | | | |
| Phenyl Trimethicone | 15.00 | 15.00 | 0 | 0 |
| Cyclopentasiloxane | 5.00 | 5.00 | 0 | 0 |
| (and) | | | | |
| Trimethylsiloxysilicate | | | | |
| Octylmethoxy- | 7.00 | 7.00 | 0 | 0 |
| cinnamate | | | | |
| Octocrylene | 8.00 | 8.00 | 0 | 0 |
| Cyclopentasiloxane | 47.00 | 0 | 80.00 | 0 |
| 1,1,1,3,5,5,5- | 0 | 47.00 | 0 | 80.00 |
| heptamethyl-3- | | | | |
| ethyltrisiloxane (ETS) | | | | |
| Octylsalicylate | 0 | 0 | 20.00 | 20.00 |

When spraying Comparative Example I and Example I onto surface, Comparative Example I shows clear signs of run-down, while Example I does not, as exemplified in FIGS. I and II in the drawings section below. Comparative Example I has a run-down on the above-identified scale of 2. Example I has run-down on the above-identified scale of 0.

As can be clearly seen, the formula with cyclopentasiloxane starts to run-down, while the formula with ETS stays in place. Here in both cases, a well primed pump spraying system was used once and applied from 10.16 centimeters away from the substrate which was notebook paper.

In order to accommodate for the possibility that the ETS containing formulations do not just not run down because they deposit less materials on the top of the substrate surface, and in order to eliminate the effect of variations from spray to spray, the following control experiments were run:

From 10.16 centimeters away, the above experiment was repeated, aiming at lined and previously carefully weighed out notepad paper. Five sprays of each of the formulations Comparative Example I & Example I were delivered, and the so delivered mass of material was obtained by gravimetry:

### Comparative Example I

0.581 grams (g) was deposited with five sprays=>0.581 g/5=0.1162 g per spray deposited. Since a run-down phenomena could also depend on the volume deposited, these results were corrected for the density of the formula, which was determined to be 0.9777 g/ml, using a Mettler/Paar DMA46 Calculating Digital Density Meter=>0.1162 g/0.9777 g/ml=0.1189 ml per spray deposited.

### Example I

0.622 g was deposited with five sprays=>0.622 g/5=0.1244 g per spray deposited. With the density determined as described above, the following volume was deposited: 0.1244 g/0.9107 g/ml=0.1366 ml per spray deposited.

In addition, all experiments were carried out with different spray pumps of the same type (Qosmedix Corporation, Edgewood, N.Y., United States white spray mister with clear overcap Model #29717, spray pump made of polypropylene and bottle is made of polyvinylchloride) to eliminate any possible effect of the pump itself.

Clearly, as can be seen from the above, the ETS formulas do not deposit less than the cyclopentasiloxane formulas, hence the no-run-down effect is genuinely due to the beneficial presence of the ETS.

Spraying Comparative Example I onto hydrated vitro-skin and measuring the resulting sun protection factor using a Labsphere UV1000 Light Simulator yields a sun protection factor of 11, while Example I gives a sun protection factor of 13 under the same conditions.

This serves as a principal example that use of trisiloxanes disclosed herein, as exemplified but not limited to 1,1,1,3,5,5,5-heptamethyl-3-ethyltrisiloxane (ETS), delivers measureable enhanced properties in various sprayable applications.

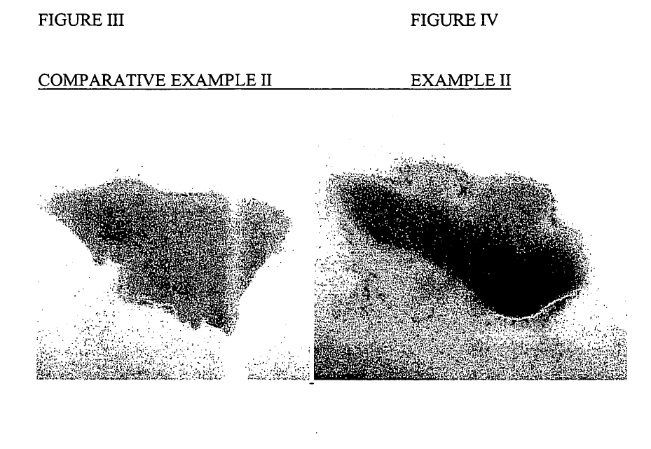

FIGS. 3 and 4 in the drawings section below shows that this no-run-down-effect when using ETS is not formulation dependant, but rather a generic benefit of the ETS. FIGS. 3 and 4 are included to show that the ingredient ETS is the main factor responsible for the decrease in run-down in the Examples over the Comparative Examples. Comparative Example II has a run-down on the above-identified scale of 3. Example II has run-down on the above-identified scale of 0. All the examples and comparative examples herein were sprayed using a well-primed spray bottle. Comparative Example II and Example II were applied from the same distance as Example I and Comparative Example I. All of the data in Table 1 was weight percent data based on the total weight of the trisiloxane-containing composition in each of the respective examples and comparative examples. Example II and Comparative Example II were applied using the same number of sprays as indicated in Example I and Comparative Example I.

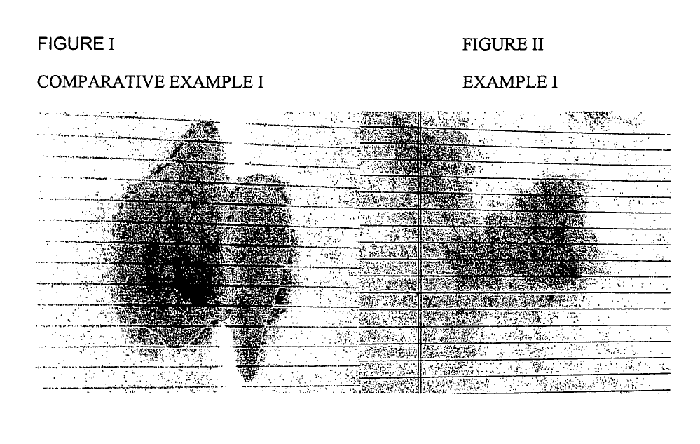

Here once again, the formula with cyclopentasiloxane does show significant run-down, while the one with ETS stays in place. FIGS. 1-4 are xerographic reproductions of photographs of patterns resulting from the application of compositions within (Examples I and II) and other compositions outside (Comparative Examples I and II) the scope of the disclosure herein, said photographs being taken shortly after (1-6 seconds) application of said compositions.

While the above description comprises many specifics, these specifics should not be construed as limitations, but merely as exemplifications of specific embodiments thereof. Those skilled in the art will envision many other embodiments within the scope and spirit of the description as defined by the claims appended hereto.

1. A method of applying a trisiloxane-containing composition to the surface of a substrate comprising:
applying to a surface of a substrate a composition comprising a trisiloxane of the general formula (1):
MDM (1)
wherein
M=R^{A}R^{B}R^{C}SiO_{1/2},
D=R^{D}R^{F}SiO_{1/2}
in which R^{D} and R^{F} each independently is a monovalent, divalent or trivalent hydrocarbon radical possessing from 2 to 8 carbon atoms or a monovalent, divalent or trivalent hydrocarbon radical possessing from one to sixty carbon atoms other than from 2 to 8 carbon atoms, provided that at least one of R^{D} and R^{F} is a monovalent, divalent or trivalent hydrocarbon radical possessing from 2 to 8 carbon atoms, and R^{A}, R^{B}, R^{C} each independently is a monovalent, divalent or trivalent hydrocarbon radical possessing from one to sixty carbon atoms other than from 2 to 8 carbon atoms.

2. The method of claim 1 where R^{D} and/or R^{F} is a monovalent, divalent or trivalent hydrocarbon radical possessing from 2 to 6 carbon atoms.

3. The method of claim 1 where R^{D} and/or R^{F} is a monovalent, divalent or trivalent hydrocarbon radical possessing from 2 to 4 carbon atoms.

4. The method of claim 1 where R^{D} and/or R^{F} is ethyl.

5. The method of claim 1 where R^{D} is ethyl and R^{F} is methyl.

6. The method of claim 1 where said trisiloxane is selected from the group consisting of ethyl trisiloxanes, octyltrisiloxanes, hexyltrisiloxanes and combinations thereof.

7. The method of claim 1 where said trisiloxane is selected from the group consisting of 3-pentyl-1,1,1,3,5,5,5-heptamethyltrisiloxane; 1,1,1,3,5,5,5-heptamethyl-3-octyltrisiloxane; 1,1,1,3,5,5,5-heptamethyl-3-hexyltrisiloxane; 3-(1-ethylbutyl)-1,1,1,3,5,5,5-heptamethyltrisiloxane; 1,1,1,3,5,5,5-heptamethyl-3-(1-methylpentyl)trisiloxane; 1,1,1,3,5,5,5-heptamethyl-3-(1-methylpropyl)trisiloxane; 3-(1,1-dimethylethyl)-1,1,1,3,5,5,5-heptamethyltrisiloxane; 1,1,1,5,5,5-hexamethyl-3,3-bis(1-methylethyl)trisiloxane; 3-(3,3-dimethylbutyl)-1,1,1,3,5,5,5-heptamethyltrisiloxane; 1,1,1,3,5,5,5-heptamethyl-3-(3-methylbutyl)trisiloxane; 1,1,1,3,5,5,5-heptamethyl-3-(3-methylpentyl)trisiloxane; 1,1,1,3,5,5,5-heptamethyl-3-(2-methylpropyl)trisiloxane; 1,1,1,3,5,5,5-heptamethyl-3-propyltrisiloxane; 3-isohexyl-1,1,1,3,5,5,5-heptamethyltrisiloxane; 3-tert-pentyl-1,1,1,3,5,5,5-heptamethyltrisiloxane; 3-neo-pentyl-1,1,1,3,5,5,5-heptamethyltrisiloxane; 1,1,1,5,5,5-hexamethyl-3,3-dipropyltrisiloxane; 3,3-diethyl-1,1,1,5,5,5-hexamethyltrisiloxane; 3,3-dibutyl-1,1,1,5,5,5-hexamethyltrisiloxane; 3-ethyl-1,1,1,3,5,5,5-heptamethyltrisiloxane; 3-heptyl-1,1,1,3,5,5,5-heptamethyltrisiloxane; 1,1,1,5,5,5-hexamethyl-3,3-diethyltrisiloxane; 1,1,1,3,5,5,5-heptamethyl-3-butyltrisiloxane and combinations thereof.

8. The method of claim 1 wherein trisiloxane-containing composition, can contain specifically less than about 100 weight percent water based on the total weight of trisiloxane-containing composition.

9. The method of claim 1 where said trisiloxane of the trisiloxane-containing composition is present in an amount of from about 0.001 to about 99 weight percent.

10. The method of claim 1 where trisiloxane-containing composition further comprises formulation component in an amount of from about 99 to about 0.001 weight percent.

11. The method of claim 10 where said formulation component is selected from the group consisting of propellant, surfactant, emulsifier, solvent, emollient, moisturizer, humectant, pigment, colorant, fragrance, biocide, preservative, antioxidant, anti-microbial agent, anti-fungal agent, insecticide, paint, antiperspirant agent, aerosol-driver, exfoliants, alcohol, hormone, enzyme, medicinal compound, vitamin, alpha-hydroxy acid, beta-hydroxy acid and/or other hydroxy acid, retinol and its derivatives, niacinamide, skin lightening agent, salt, electrolyte, polyol, absorbing agent for ultraviolet radiation, botanical extract, organic oil, wax, film former, thickening agent, particulate filler, clay, plasticizer, occlusive, sensory enhancer, ester, resin and film formers, spherical or other optically active particle, and combinations thereof.

12. The method of claim 1 where trisiloxane-containing composition can further comprise combination of the foregoing trisiloxane of the general formula (1) with other types of silicones selected from the group consisting of silicone oil, silicone gum, silicone solid, silicone emulsion, silicone resin, silicone copolyol, alkyl silicones, silicone waxes, silicone amines, silicone ammonium salts, quaternary silicone amines, spherical silicones, silicone gels, silicone crosspolymer networks, polyether silicones, and combinations thereof.

13. The method of claim 1 where trisiloxane-containing composition can further comprise silicone fluid other than said trisiloxane of the general formula (1).

14. The method of claim 13 where trisiloxane of trisiloxane-containing composition and/or silicone fluid other than said trisiloxane of the general formula (1) can be made using a precious metal catalyst.

15. The method of claim 1 where applying the trisiloxane-containing composition comprises using a spray that employs at least one spraying, dispensing or diffusing or mistyfying process.

16. The method of claim 1 where applying the trisiloxane-containing composition comprises using a spray selected from the group consisting of a fragrance and lotion pump, fine mist sprayer, a specialty pump, an anti-clog pump, a spray-through cap, a nozzle, an airless pump, an airless dispenser, a dual dispensing pump, a fine mist pump, a trigger pump, a foam pump, a trigger sprayer, an aerosol sprayer, a squeeze roamer and combinations thereof to provide an application of said spray.

17. The method of claim 16 where applying the trisiloxane-containing composition further comprises spraying from about 0.001 milliliters to about 50 milliliters per application of said spray.

18. The method of claim 16 where applying the trisiloxane-containing composition further comprises using said spray at a distance of from about 0.01 to about 10,000 centimeters from said surface of said substrate.

19. The method of claim 1 where said trisiloxane-containing composition comprises other than a cosmetic spray, a gel, or an emulsion.

20. The method of claim 1 wherein the applied trisiloxane-containing composition exhibits at least one of reduced run-down, better coverage, improved actives distribution and/or performance, rapid drying, less over-wetting, easy formulation, non-tackiness, improved spreadability, improved homogeneity, improved organic compatibility, reduced skin irritation, improved active delivery, improved skin feel, enhanced SPF, less or more shine as desired, smaller droplet size, less mechanical stress on the spraying system employed properties that are improved over a sprayable composition containing other than the composition of general formula (1).

21. The method of claim 20 where said sprayable composition containing other than the composition of general formula (1) is a volatile siloxane.

22. The method of claim 21 where said volatile siloxane is a cyclic siloxane.

23. The method of claim 20 wherein the run-down exhibits little downward extension of incident spray on the surface of the substrate.

24. The method of claim 20 where the improvement in said properties is synergistic.

25. The method of claim 20 where said properties are the result of a synergistic effect produced by using a synergistic component selected from the group consisting of emulsifier; preservative; polymer; thixotropic and non-thixotropic film former; sunscreening agent; sunblocking agent; colorant; pigment; fragrance; active ingredient; retinol and its derivatives; niacinamide; tocopherol and/or its derivatives; skin lightening agent; humectant; occlusive; sensory enhancer; ester; other resin and film former; spherical or other optically active particle; other silicone; emollient; surfactant; and combinations thereof.

26. The method of claim 1 where applying trisiloxane-containing composition can comprise where trisiloxane-containing composition is used in applications where it is desired to have any coating related advantageous property.

27. The method of claim 1 where trisiloxane-containing composition can be used in markets and industries selected from the group consisting of aerospace, agriculture, food & beverage, tissue & non-wovens, automotive, tires, coatings, inks, paints, construction, consumer, electronics, semiconductors, healthcare, homecare, industrial assembly, industrial cleaning, mold making, packaging and labeling, power & electric distribution, process industries, transportation, pool & spa, motorcycles, personal care and combinations thereof.

28. The method of claim 27 where the personal care markets and industries can comprise using the trisiloxane-containing composition in a personal care application selected from the group consisting of deodorant, antiperspirant, antiperspirant/deodorant, shaving product, skin lotion, moisturizer, toner, bath product, cleansing product, shampoo, conditioner, combined shampoo/conditioner, mousse, styling gel, hair spray, hair dye, hair color product, hair bleach, waving product, hair straightener, nail polish, nail polish remover, nail cream and/or lotion, cuticle softener, sunscreen, insect repellent, anti-aging product, lipstick, foundation, face powder, eye liner, eye shadow, blush, makeup, mascara, moisturizing preparation, body and hand preparation, skin care preparation, face and neck preparation, tonic, dressing, hair grooming aid, aerosol fixative, fragrance preparation, aftershave, make-up preparation, soft focus application, night and day skin care preparation, non-coloring hair preparation, tanning preparation, synthetic and non-synthetic soap bar, hand liquid, nose strip, non-woven application for personal care, baby lotion, baby bath and/or shampoo, baby conditioner, shaving preparation, cucumber slice, skin pad, make-up remover, facial cleansing product, cold cream, sunscreen product, spritz, paste mask and/or mud, face mask, cologne and toilet water, hair cuticle coat, shower gel, face and body wash, personal care rinse-off product, gel, foam bath, scrubbing cleanser, astringent, nail conditioner, eye shadow stick, powder for face or eye, lip balm, lip gloss, hair care pump spray and other non-aerosol spray, hair-frizz-control gel, hair leave-in conditioner, hair pomade, hair de-tangling product, hair fixative, hair bleach product, skin lotion, pre-shave and pre-electric shave, anhydrous cream and/or lotion, oil/water, water/oil, multiple and macro and micro emulsion, pickering emulsion, multiple-phase emulsion, water-resistant cream and/or lotion, anti-acne preparation, mouth-wash, massage oil, toothpaste, clear gel and stick, ointment base, topical wound-healing product, aerosol talc, barrier spray, vitamin and anti-aging preparation, herbal-extract preparation, bath salt, bath and body milk, hair styling aid, hair-, eye-, nail- and skin-soft solid application, controlled-release personal care product, hair conditioning mist, skin care moisturizing mist, skin wipe, pore skin wipe, pore cleaner, blemish reducer, skin exfoliator, skin desquamation enhancers, skin towelette and/or cloth, depilatory preparation, personal care lubricant, nail coloring preparation, drug delivery system for topical application of medicinal composition that is to be applied to the skin and combinations thereof.

29. The method of claim 28 where personal care application is a suncare formulation that can further comprise sunscreen agent selected from the group consisting of UV blocking or UV absorbing sunscreening agent, and combinations comprising at least one of the foregoing sunscreening agents.

30. The method of claim 28 where personal care application can further comprise emulsifier selected from the group consisting of non-ionionic biopolymer blends, polyethoxylated emulsifiers, modified polyacrylates, oils, animal and/or synthetic waxes hydrocarbon gels or bentone type gels, organosilicone emulsifiers and combinations thereof.

31. The method of claim 1 where trisiloxane-containing composition can be used in a product form that can be anhydrous to non-anhydrous.

32. The method of claim 31 where said product form can be a simple solid, a particle, a solution, a gel-network-stabilized phase and combinations thereof.

33. The method of claim 1 where trisiloxane-containing composition can be used in a product forms selected from the group consisting of a gel, an emulsion, a dispersion, a sludge, a powder, a bar, a spray, a spritz, a mousse, a stick, a balm, a foundation and combinations thereof.

34. The method of claim 1 where trisiloxane-containing composition can further comprise plasticizer selected from the group consisting of lecithin, polysorbates, dimethicone copolyol, glycols, citrate esters, glycerin, dimethicone and combinations thereof.
